(19) United States
(12) Patent Application Publication
(10) Pub. No.: US 2016/0015969 A1
(43) Pub. Date: Jan. 21, 2016

(54) METHOD AND APPARATUS FOR FOR CONTROLLED DISSIPATION OF RADIATED ENERGY EMISSION

(71) Applicants: Serdar Kiykioglu, Plano, TX (US); Terry Daglow, Houston, TX (US)

(72) Inventors: Serdar Kiykioglu, Plano, TX (US); Terry Daglow, Houston, TX (US)

(21) Appl. No.: 14/722,137

(22) Filed: May 26, 2015

Related U.S. Application Data
(60) Provisional application No. 62/002,924, filed on May 26, 2014.

(51) Int. Cl. A61N 1/08; H05K 9/00

(52) U.S. Cl. CPC A61N 1/08; H05K 9/0081

(57) ABSTRACT

Disclosed herein are devices and methods for shielding certain medical conductors, such as neurotherapy leads from MRI energy. Preferred embodiments comprise a heat sink; a first antenna coil extending from the heat sink and wound around the longitudinal axis of the therapeutic conductor in a first longitudinal direction; and a second antenna coil extending from the heat sink and wound around the longitudinal axis of the therapeutic conductor in a second longitudinal direction.

### CROSS REFERENCE TO RELATED APPLICATIONS

This is a non-provisional application claiming priority to U.S. Provisional Application No. 62/002,924, filed May 26, 2014, which is incorporated herein by reference.

### BACKGROUND

The disclosed inventions are broadly related to leads for neurostimulation therapy, and more particularly, related to limiting the induction of currents in electro stimulation leads during MRI procedures.

Neurostimulation therapy involves the stimulation of nerve tissues with electric pulses to treat a medical condition. Examples of neurostimulation therapies include spinal cord stimulation, deep brain stimulation, cortical stimulation, cochlear nerve stimulation, peripheral nerve stimulation, vagal nerve stimulation, and sacral nerve stimulation, among others. In the example of spinal cord stimulation, electrical pulses are used to stimulate nerves in the spine to help control chronic pain. The electrical pulses can induce a sensation of numbness and mask the transmission of pain sensations.

Neurostimulation systems generally comprise a pulse generator and one or more leads connecting the pulse generator to the tissue being stimulated. The pulse generator is usually implanted within a subcutaneous pocket created under the skin by a physician. The leads typically comprise a conductive wire surrounded by a insulating polymer. Electrodes on a distal end of the leads are electrically connected to the lead wires and configured to deliver the electrical pulses to the nerve tissue.

There are concerns related to the compatibility of neurostimulation systems with magnetic resonance imaging (MRI). MRI creates internal images of the human body using oscillating magnetic and RF fields. Wires, such as those used in neurostimulation leads can act as antennae within the oscillating field, thereby converting some of the energy into induced electric currents. These induced currents can cause significant heating in parts of a neurostimulation system which can potentially damage surrounding tissue. As a result, most neurostimulation systems are contraindicated for MRI.

An antenna is a transducer designed to transmit electromagnetic energy to a medium or to receive electromagnetic energy from a medium and deliver to a load. Two major factors associated with radio antenna design are the antenna resonant point or center operating frequency and the antenna bandwidth or the frequency range over which the antenna design can operate.

An RF antenna is a form of tuned circuit consisting of inductance and capacitance, and as a result it has a resonant frequency. Inductance of an antenna is determined by the thickness of the wire it is made of, the length, and the construction of the antenna. Capacitance is determined by the surface area of the antenna and the dielectric constant of the medium in which the antenna is operating within.

Antennas are designed to resonate at the required specific frequency, meaning that there is a limited bandwidth over which an antenna can operate efficiently. At the resonance frequency, the capacitive and inductive reactance values cancel each other out. At this point the RF antenna appears purely resistive, and the resistance is determined by the combination of the loss resistance and the radiation resistance.

Antenna size is determined by the required operating frequency. Therefore, by changing the inductance and capacitance antenna size can be varied for any given operating frequency.

Antenna type also determines the bandwidth. A folded dipole has a wider bandwidth than a non-folded one. Therefore if the desired operating frequency band is narrow a non-folded dipole may be desired.

Current in a receiving conductor is created if an electric field is induced in that conductor. That electric field can be created either by the received electric field component or by the magnetic field component or a combination of both. So when a conductor is exposed to radiated emission, current flows in the conductor and may cause heating. If the radiated energy can be picked up and delivered to an electrical load in a controlled manner, then the effects of the heating can be controlled by dissipating it in a controlled manner.

### SUMMARY

This application teaches an apparatus and method for an antenna and electrical load embodiment to pick up radiated emission of a frequency or a plurality of frequencies, and convert the energy into heat for controlled dissipation of the said energy.

### BRIEF DESCRIPTION OF THE DRAWINGS

FIG. 1 shows a therapy lead protected by one embodiment of MRI shields as disclosed herein.

FIG. 2 shows a closer view of an MRI shield for a therapy lead.

FIG. 3 shows a neurotherapy system incorporating disclosed MRI energy dissipation inventions disclosed herein.

### DETAILED DESCRIPTION OF THE PREFERRED EMBODIMENTS

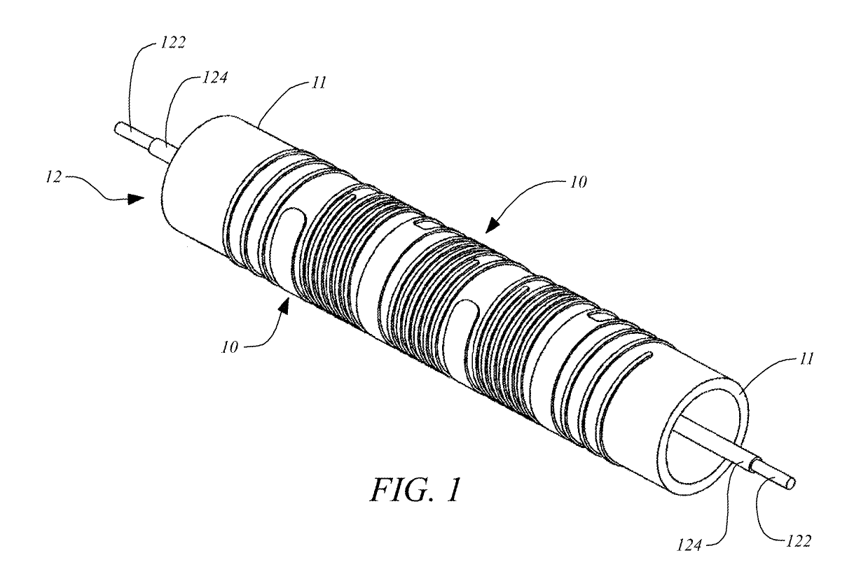

Referring to FIG. 1, a plurality of MRI shields 10 surrounds a catheter tube 11, which encloses a therapy lead 12. Therapy lead 12 comprises a therapy conductor 122 surrounded by a conductor insulation 124. Therapy conductor 122 preferably comprises a flexible, electrically-conductive material, such as copper wire.

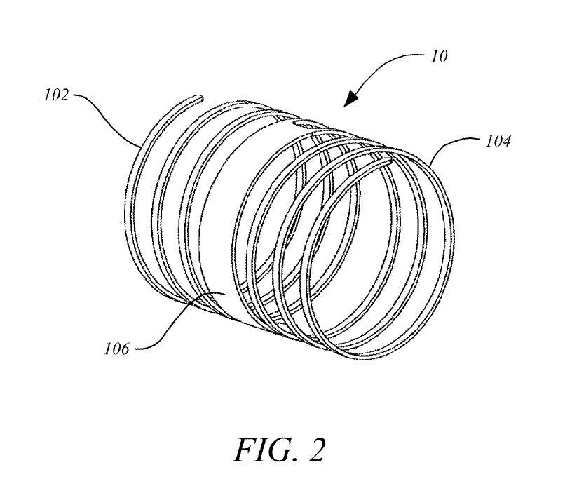

FIG. 2 shows a larger view of one unit of MRI shield 10. MRI shield 10 preferably comprises first antenna 102, second antenna 104, and heat sink 106. Heat sink 106 is positioned between first antenna 102 and second antenna 104 and is electrically connected to each of them. First antenna 102 and second antenna 104 are preferably configured as coils extending from opposite sides of heat sink 106. Preferably, first antenna 102 and second antenna 106 are wrapped in opposite twist directions (i.e. so that one mirrors the other).

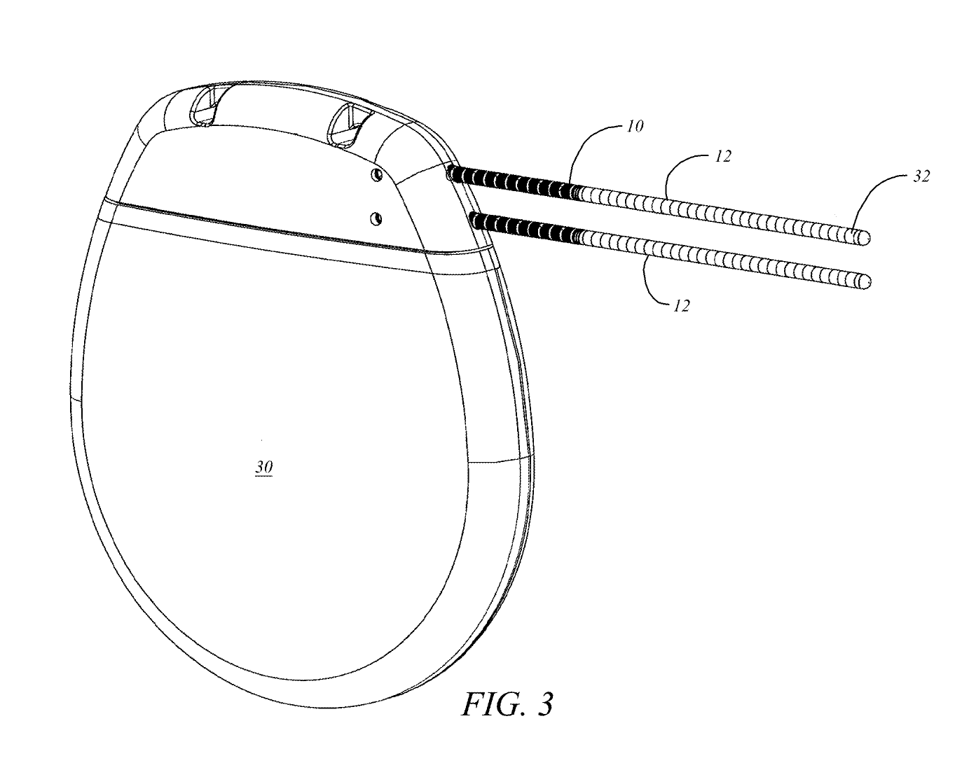

FIG. 3 illustrates the major components of a neurostimulation system. The system comprises a pulse generator 30, therapy lead 12, electrodes 32, and MRI shields 10. MRI shields 10 preferably extend along at least an initial portion of therapy lead 12 extending from pulse generator 30.

Example: For an alternating magnetic field, with 64 MHz operating frequency, a folded or a non-folded dipole antenna is envisioned, with dimensions defined by the wavelength at 64 MHz, which vary based on the medium in which the antenna is intended to operate. For instance, dielectric constant of free space is 1, however, if the antenna is operating in a medium other than free space, one would know that the relative dielectric constant of the medium may be larger than 1, thus the antenna dimensions will be smaller.

In this example, it is envisioned that the antenna is constructed as a center feed dipole, and the dipole is delivering picked up energy to a resistive electrical load, thereby converting the electromagnetic energy into heat.

If a plurality of the said dipoles can be constructed where and the center of the dipoles are spatially separated, then the energy converted into heat at the resistive electrical load can be distributed, thereby dissipating the electromagnetic energy uniformly over a defined area in three dimensional space, thus preventing non uniform hot spots.

This invention can prevent a conductor structure from picking up electromagnetic energy which may cause unintended heating of the conductor, by acting as an energy shield. In this application the energy pick up and controlled heat dissipation antenna can be constructed over the conductor to be protected.

In order to pick up incident energy on the antennae without constraint by the direction of radiation, one may wind the antenna spirally over the conductor to be protected.

This construction may further improve the affectivity and efficiency of this invention by the increased inductance of the antennae, thereby enabling shorter antenna designs. This in turn enables more antennae and larger number of resistive loads to be constructed thereby making heat distribution more uniform.

This above mentioned antenna designs, electrical load, construction methods, and application are examples only and this invention is not limited those described.

Although representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure that processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

I claim:

1. An electromagnetic shield for an elongate therapeutic conductor with a longitudinal axis comprising:
a heat sink;
a first antenna coil extending from the heat sink and wound around the longitudinal axis of the therapeutic conductor in a first longitudinal direction; and
a second antenna coil extending from the heat sink and wound around the longitudinal axis of the therapeutic conductor in a second longitudinal direction.

2. The electromagnetic shield of claim 1 wherein the heat sink comprises a partial cylinder configured to at least partially surround the therapeutic conductor.

3. The electromagnetic shield of claim 1 where in the heat sink, the first antenna coil, and the second antenna coil comprise a single part.
